(19) United States
(12) Patent Application Publication
(10) Pub. No.: US 2026/0078328 A1
(43) Pub. Date: Mar. 19, 2026

(54) MICROPHYSIOLOGICAL SYSTEM

(71) Applicant: UNIVERSITE DU LUXEMBOURG, Esch-sur-Alzette (LU)

(72) Inventors: Paul WILMES, Bettembourg (LU); Catherine SEDRANI, Wormeldange (LU); Kacy GREENHALGH, Luxembourg (LU); Léa GRANDMOUGIN, Rodemack (FR); Mara LUCCHETTI, Rumelange (LU); Audrey FRACHET BOUR, Metz (FR); Kris VER DONCK, Herent (BE); Frederik MORTIER, Merendree (BE); Sven VAN GEYT, Gentbrugge (BE)

(21) Appl. No.: 19/130,155

(22) PCT Filed: Nov. 17, 2023
(86) PCT No.: PCT/EP2023/082179

§ 371 Date: May 15, 2025

(30) Foreign Application Priority Data
Nov. 17, 2022 (LU) LU503075

(51) Int. Cl. C12M 1/00; C12M 1/34; C12M 3/00

(52) U.S. Cl. CPC C12M 23/38; C12M 23/22; C12M 23/26; C12M 23/46; C12M 23/48; C12M 23/50; C12M 41/12

(57) ABSTRACT

The disclosure relates to a microphysiological system comprising a frame with a base (7) exhibiting a surface (9) for receiving a cell culture support, a lid for closing the microphysiological system, and locking means fastening the frame and the lid when said microphysiological system is closed, the microphysiological system is remarkable in that the base (7) is disposed within the frame on a support plate (17) displaceable between a high position when the microphysiological system is open and a low position when the microphysiological system is closed and in that the support plate (17) is movable vertically between its high position and low position with elastic means (19) configured to bring the support plate (17) back to the high position.

### FIELD OF THE DISCLOSURE

The present disclosure relates to a microphysiological system.

### BACKGROUND OF THE DISCLOSURE

Microphysiological systems serve as in vitro models for bone, cartilage, brain, gastrointestinal tract, lung, liver, microvasculature, reproductive tract, skeletal muscle, and skin. They are therefore used in biology, medicine, pharmacology, physiology, and toxicology to mimic these important organs of living organisms.

Microphysiological systems comprise generally channels separated by a permeable or semi-permeable membrane, the channels being arranged between a base and a lid of the microphysiological system. Thus, a cell culture layer mimicking the vascular circuit is generally arranged in a first channel disposed on a surface of a base. Such a culture layer is for example a peripheral blood mononuclear cell (PBMC) culture layer. To be maintained in function, such a cell culture layer receives oxygen, nutrients, and other growth factors via cell culture medium that is perfused through the system via perfusion holes placed on the bottom of the surface of the base, and the top of the surface of the lid. On top of the first channel, there is a second channel comprising an epithelium layer, to mimic the cell walls of a particular organ that is mimicked in the microphysiological system. For example, in the case where the intestinal epithelial barrier is to be mimicked, Caco-2 cells can form the epithelium layer. On top of the epithelial layer, a microbial layer is presented, usually in a third channel. This microbial layer presents the microbiome associated with the body site that is represented in the microphysiological system, and comprises among others, bacteria. The microbiome is there to enable representative interaction between the human or mammalian cell layers and the microbiome in the microphysiological system

Finally, on top of the third channel, there is generally a fourth channel for conducting an inert gas, such as nitrogen, to create an anaerobic environment, mimicking the conditions in the intestinal tract. Thus, tubings must be set up in the microphysiological system to, on one hand, direct inert gases, such as nitrogen, to provide anaerobic conditions and, on the other hand, through microfluididic perfusion of liquids through the system, bring elements that are required to maintain sustainable ecological conditions for the particular organ that is studied in the microphysiological system. One example of such elements is a component to establish a required pH.

US 2019/0345431 describes a microphysiological system comprising a base having a recessed surface for receiving a cell culture support layer, a lid having a stepped surface configured to exert a contact force to a top layer of a plurality of cell culture support layers during use and a clamp operably connected to each of the base and the lid. The clamp has an engagement mechanism generating the contact force and fluidically sealing the plurality of cell culture support layers. A uniform contact pressure exerted on the top layer of the plurality of the cell culture support layer can be generated by using a plurality of clamps that are distributed around the perimeter of the base and the lid.

However, in operation, leakage can occur since it is sometimes difficult to assemble the plurality of layers between the base and the lid. Indeed, upon closure, the force that is applied on each clamp is exerted on the surrounding of the clamp that is activated. Upon many operations of assembling and disassembling the microphysiological system, as well as sterilising the system under conditions of high pressure (between 40 and 110 kPa) and high temperature (between 100° C. and 120° C.), the risk occurring that the base and the lid of the microphysiological system slightly move with respect to each other is also to be considered when exerting locally a force. The resulting leakage impedes the working conditions of the microphysiological system since it can result not only in a loss of the required elements that are brought to the system during operation but also in the subsequent contamination by external elements of the organ that is mimicked in the microphysiological system.

The present disclosure aims to provide a microphysiological system in which the problem of leakage is avoided or at least reduced. The present disclosure aims to provide a microphysiological system in which the problem of leakage is avoided or at least reduced and that is also robust and/or easy to clean.

### SUMMARY OF THE DISCLOSURE

According to a first aspect, the disclosure provides a microphysiological system comprising a frame with a base exhibiting a surface for receiving a cell culture support, a lid for closing the microphysiological system, and locking means fastening the frame and the lid when said microphysiological system is closed, the microphysiological system is remarkable in that the base is disposed within the frame on a support plate displaceable between a high position when the microphysiological system is open and a low position when the microphysiological system is closed and in that the support plate is movable vertically between its high position and low position with elastic means configured to bring the support plate back to the high position.

Surprisingly, it has been found that the use of a support plate that is movable vertically within the frame, and that can be brought back to the high position by the elastic restoring force, can distribute the force all along the base of a microphysiological system. In fact, upon assembly of the microphysiological system, when the locking means are sealing the lid to the frame, the contact force is exerted not only on the surroundings of the locking means but is spread on the support plate and therefore ensures a uniform sealing of the lid and the frame together, which encloses the different layers (for example, the cell culture layer mimicking the vascular circuit, the epithelium layer and the microbiome layer). The support plate is movable vertically and the elastic means (such as one or more coil springs) are configured to bring back the movable plate to its high position. In other words, the support plate is configured to push the base up, through the elastic means, so it exerts a controlled and adjustable amount of pressure against the lid. This configuration allows for a gas-tight and a liquid-tight sealing of the different layers together. The risk of leakage is therefore avoided or at least considerably reduced and subsequently, the risk of contamination of the different layers as well.

In particular, the disclosure provides a microphysiological system comprising a frame with a base exhibiting a surface for receiving a cell culture support, a lid for closing the microphysiological system, and locking means fastening the frame and the lid when said microphysiological system is closed, the microphysiological system is remarkable in that the base is disposed within the frame on a support plate displaceable between a high position when the microphysiological system is open and a low position when the microphysiological system is closed, in that the support plate is movable vertically between its high position and low position with elastic means configured to bring the support plate back to the high position, and in that the base shows at least two pins oriented vertically and the lid comprises at least two ports in which said at least two pins are arranged when said microphysiological system is closed.

It has been indeed found that at least two pins ensure that the assembly between the frame and the lid is correctly performed, so as providing a correct sealing between the lid and the frame when the microphysiological system is closed.

Advantageously, the base shows at least two edges facing each other, and the base presents one of said at least two pins oriented vertically along each of said two edges facing each other.

More preferably, said at least two pins oriented vertically are shifted between each other.

Advantageously, the base and the lid show each at least one access aperture.

With preference, the locking means are or comprise at least two clamps.

Advantageously, the elastic means are or comprise one or more springs, such as one or more coil springs.

With preference, the elastic means are arranged centrally with respect to the support plate.

Advantageously, the support plate comprises one or more positioning means cooperating with the elastic means and/or with the frame to ensure a vertical movement of the support plate with respect to the frame. With preference, the positioning means of the support plate comprise one or more feet.

For example, the elastic means comprise one or more coil springs arranged or coiled around the one or more feet of the support plate, preferably at least one coil spring is arranged or coiled around one foot.

For example, the frame comprises one or more passages forming guiding means cooperating with the elastic means and/or the support plate to ensure a vertical movement of the support plate with respect to the frame.

For example, the frame has a structure presenting at least two vertical panels facing one another and connected by a connecting structure wherein the connecting structure presents one or more passages forming guiding means.

For example, the frame has a structure presenting at least two vertical panels facing one another and connected by a connecting structure wherein the connecting structure presents one or more passages forming guiding means and the lid for closing the microphysiological system has a similar structure with at least two lateral elements intended to cover at least a part of the top of the at least two vertical panels of the frame and a central element wherein the central element presents one or more passages therein. With preference, the two lateral elements and the central element of the lid are integral.

Advantageously, the locking means, fastening the frame and the lid, comprises attachment means cooperating with hooks, wherein the hooks are formed by an outgrowth of the lid or of the frame so as to be integral with it.

Advantageously, the microphysiological system further comprises one or more pre-setting tools of the elastic means configured for adjusting the level of the high position of the support plate and/or the pressure generated between the base and the lid. Thus, the force exerted by the elastic means can be adjusted and pre-set to apply the correct pressure for the functioning of the system

Advantageously, the lid for closing the microphysiological system comprises one or more openings. With preference, at least a part of the one or more openings of the lid are covered with at least one optically transparent sheet made of an optically transparent plastic material.

Advantageously, the structure of the frame and the structure of the lid are in a metallic material. For example, the metallic material is steel.

Advantageously, the support plate is made of an optically transparent plastic material.

For example, the optically transparent plastic material is polycarbonate or cyclic olefin copolymer, more preferably polycarbonate.

Advantageously, the surface exhibited by the base for receiving a cell culture support is a recessed surface and the lid for closing the microphysiological system has a stepped surface facing the surface of the base.

In a preferred embodiment, the microphysiological system comprises a first electrical heating mat under the surface for receiving a cell culture support and a second electrical heating mat under the lid for closing the microphysiological system. With preference, the microphysiological system further comprises a first conduct intended to be filled with a liquid and arranged between the first electrical heating mat and the surface for receiving a cell culture support and a second conduct intended to be filled with a liquid and arranged under the second electrical heating mat. For example, the second conduct intended to be filled with a liquid is under the second electrical heating mat and facing the cell culture support of the base.

In a preferred embodiment, the microphysiological system further comprises a cell culture support arranged on the surface, wherein the cell culture support is imbricated within a gasket.

For example, the microphysiolgical system further comprises a cell culture support arranged on the surface, wherein the cell culture support is an arrangement of at least two or more channels, each channel forming a chamber and each channel being stacked one on the other.

More preferably, the cell culture support comprises at least four channels.

With preference, the base exhibiting a surface for receiving a cell culture support shows at least two pins oriented vertically and each of the channels comprises at least two holes in which said at least two pins are arranged.

For example, the base shows at least two edges facing each other, and the base presents one of said at least two pins oriented vertically along each of said two edges facing each other, and each of the channels comprises at least two holes in which said at least two pins are arranged when said microphysiological system is closed.

More preferably, said at least two pins oriented vertically are shifted between each other, and each of the channels comprises at least two holes in which said at least two pins are arranged when said microphysiological system is closed. For example, the channels are arranged one above the other in staggered manner, preferably in staggered rows.

With preference, each channel is a straight channel.

With preference, each channel comprises entrance means and exit means, said entrance means and exit means providing fluidic access to said channel and being directed through the lid and/or the base of the frame. Advantageously, each entrance means and exit means are fitted with a taper with a length being equal to or inferior to the thickness of the lid and/or the base of the frame. For example, the taper is a Luer-type taper or a Barb-type taper, more preferably a Luer-type taper.

Advantageously, the lid for closing the microphysiological system comprises at least one threaded opening per channel, each threaded opening being closed with a screw during the operation of the microphysiological system.

Advantageously, the lid for closing the microphysiological system comprises at least one optode having a width that is equal to or smaller than the maximum width of the chamber formed by the channel. For example, the one or more optodes are selected from an oxygen optode, a carbon dioxide optode or a pH optode.

According to a second aspect, the disclosure provides for the use of the microphysiological system according to the first aspect for culturing cells. For example, the use of the microphysiological system according to the first aspect is for modelling the gastrointestinal tract.

### DESCRIPTION OF THE FIGURES

FIG. 1: Cross-sectional view of the microphysiological system according to the disclosure.

FIG. 2: Representation of an open-structure of the frame of a microphysiological system according to the disclosure.

FIG. 3: Representation of a lid for closing a microphysiological system having an open structure according to the disclosure.

FIG. 4: Representation of a closed-structure of the frame of a microphysiological system according to the disclosure.

FIG. 5: Representation of a lid for closing a microphysiological system having a closed structure according to the disclosure.

FIG. 6: Representation of a lid with shifted ports for closing a microphysiological system having a closed structure according to the disclosure.

FIG. 7: Representation of a support plate that is used in the microphysiological system according to the disclosure.

FIG. 8: Cross-sectional view of the part between the lid and the base of a microphysiological system according to the disclosure.

### DETAILED DESCRIPTION OF THE INVENTION

### For the Purpose of the Invention, the Following Definitions Are Given.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises”and “comprised of”also include the term “consisting of”.

The terms “high” and “low”, or “top” and “under”, as used herein will be understood according to their usual definition, in which the terms “low” and “under” indicate a greater proximity to the ground in the vertical direction than respectively the terms “high” and “top”. Similarly, these terms must be understood in the normal working conditions of the microphysiological system.

The recitation of numerical ranges by endpoints includes all integer numbers and, where appropriate, fractions subsumed within that range (e.g., 1 to 5 can include 1, 2, 3, 4, 5 when referring to, for example, a number of elements, and can also include 1.5, 2, 2.75 and 3.80, when referring to, for example, measurements). The recitation of endpoints also includes the recited endpoint values themselves (e.g., from 1.0 to 5.0 includes both 1.0 and 5.0). Any numerical range recited herein is intended to include all sub-ranges subsumed therein.

The particular features, structures, characteristics or embodiments may be combined in any suitable manner, as would be apparent to a person skilled in the art from this disclosure, in one or more embodiments.

A microphysiological system (1, 3) comprises a frame 5 with a base 7 exhibiting a surface 9 for receiving a cell culture support 42, a lid (11, 13) for closing the microphysiological system (1, 3), and locking means 15 fastening the frame 5 and the lid (11, 13) when said microphysiological system (1, 3) is closed. The cell culture support 42 is imbricated within a gasket (i.e., a seal) that can be received on the surface 9 of the base 7 and is usually an arrangement of at least two or more channels (43, 45, 47, 49), each channel being stacked one on top of the other, and being preferably separated from another channel by a permeable or a semi-permeable membrane. Such membrane or semi-permeable membrane is made in an optically transparent material, being for example polycarbonate or cyclic olefin copolymer, preferably polycarbonate. Biologically compatible pressure double-sided sensitive adhesive is present to bind each channel to either side of the permeable or semi-permeable membrane.

For example, the permeable polycarbonate membranes show a diameter of 70 mm and are nanoporous with a thickness of 6 μm (e.g., Advantec MFS Inc., Dublin, CA, USA).

For example, the semi-permeable polycarbonate membranes show a diameter of 46 mm and are nanoporous with a thickness of 6 μm (e.g., Advantec MFS Inc., Dublin, CA, USA).

For example, the double-sided sensitive adhesive is commercially available at Adhesives Research, Glen Rock, PA, USA).

To ensure optimal protection against leakage during the operation of the microphysiological system (1, 3), the microphysiological system (1, 3) of the present disclosure is remarkable, as shown in the cross-section of FIG. 1, in that the base 7 is disposed within the frame 5 on a support plate 17 displaceable between a high position when the microphysiological system (1, 3) is open and a low position when the microphysiological system (1, 3) is closed and in that the support plate 17 is movable vertically between its high position and low position by means of elastic means 19 configured to bring the support plate 17 back to the high position.

Thus, upon closure of the microphysiological system (1, 3) by the user, the contact force between the lid and the frame that is coming by activation of the locking means, which are preferably two clamps, is uniformly distributed under the base 7 thanks to the support plate 17, which results in a fluid-tight sealing of the microphysiological system and subsequently of the arrangement of said at least two or more channels (43, 45, 47, 49) which is imbricated into the gasket.

Although the surface area of the support plate 17 can be smaller than the surface area of the base 7 (as in the case shown in FIG. 1), it can be advantageous that the surface area of the support plate 17 is equivalent to the surface area of the base 7 to optimize the distribution of the contact force all over the base 7.

In order to facilitate the closure of the microphysiological system (1, 3) by the user, it is preferable that the base 7 shows at least two pins oriented vertically and the lid (11, 13) comprises at least two ports 65 in which said at least two pins are arranged.

Advantageously, the base 7 shows at least two edges facing each other, and wherein the base 7 presents one of said at least two pins oriented vertically along each of said two edges facing each other. It is even more preferably that said at least two pins oriented vertically are shifted between each other. The fact that said at least two pins are shifted between each other ensures that the lid (11, 13) is correctly assembled in the right position with the frame 5, favouring subsequently the correct sealing between the lid (11, 13) and the frame 5 when the microphysiological system (1, 3) is closed. The shifting of said at least two pins between each other can be described as an arrangement of a first pin in a first position located along one first edge of the base 7 such that it does not orthogonally project onto a second position corresponding to the position of the second pin, said second position being located along one second edge of the base 7, wherein the first edge and the second edges are facing each other.

Advantageously, the base 7 and the lid (11, 13) show each at least one access aperture. For example, each access aperture of the lid (11, 13) can correspond to each access aperture of the base 7, although it is not necessary the case. Indeed, it is possible that the one or more access apertures of the base (7) are not aligned with the one or more access apertures of the lid (11, 13). The one or more access apertures of the base 7 and of the lid (11, 13) are used for connecting biological sensors, controllers, and/or fluidic inlets and outlets. For example, the base 7 and lid (11, 13) show each two access apertures.

A more particularly preferred configuration of the microphysiological system (1, 3) of the present disclosure can be described as having the base 7 disposed within the frame 5 on a support plate 17 displaceable between a high position when the microphysiological system (1, 3) is open and a low position when the microphysiological system (1, 3) is closed, wherein the support plate 17 is movable vertically between its high position and low position by means of elastic means 19 configured to bring the support plate 17 back to the high position. In addition, the base 7 shows at least two pins oriented vertically, preferably shifted between each other, and the lid (11, 13) comprises at least two ports 65 in which said at least two pins are arranged. Moreover, the base 7 and the lid (11, 13) show each at least one access aperture. In that particularly preferred configuration, a robust sealing is ensured, not only by the fact that the contact force between the lid (11, 13) and the frame 5 is uniformly distributed under the base 7 thanks to the support plate 17, but also by the fact that a poka-yoke design is present on the base 7 and the lid (11, 13) to ensure a correct orientation of this at least two elements of the microphysiological system (1, 3) for facilitating the closure and therefore the correct sealing of the microphysiological system (1, 3). In addition of the robust sealing, the one or more access apertures facilitate the handling of the microphysiological system (1, 3) by allowing it to be in constant connection with biological instruments (sensors, controllers . . . ) and/or with any kind of biological media necessary for the adequate maintenance of the cell cultures under study.

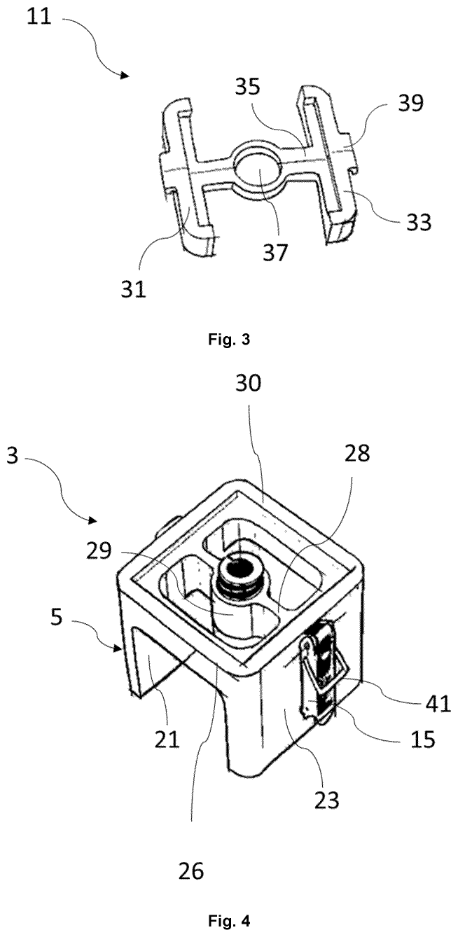

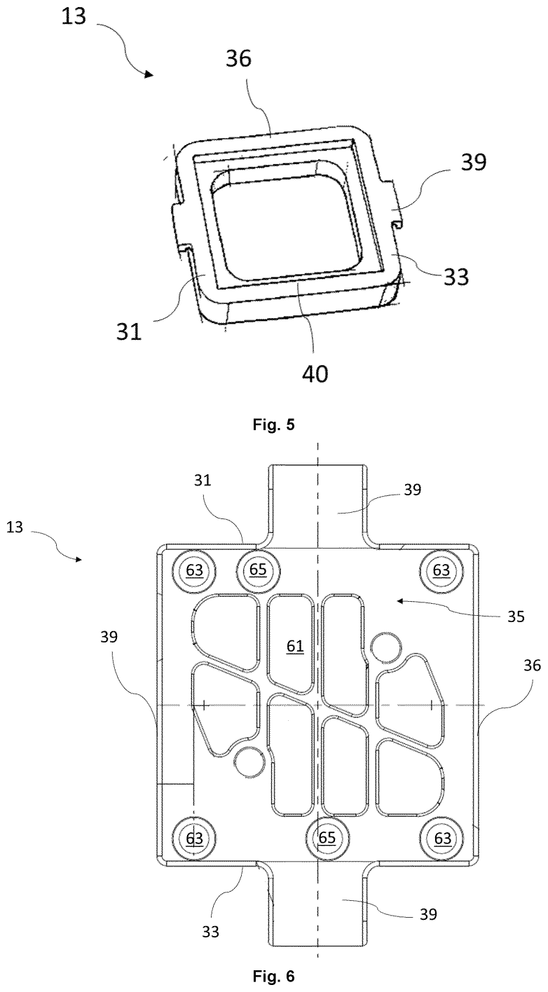

It is preferred that the locking means 15 are at least two clamps. Frame 5 can comprise at least two vertical panels (21, 23) facing one another and the locking means 15, preferably at least two clamps, are preferably disposed on each of the at least two vertical panels (21, 23) facing one another. This disposition helps the support plate 17 to foster a uniform distribution of the contact force upon closure. The locking means 15 generally comprise attachment means for them to be fixed on the walls that are to be secured together. In the case where the locking means 15 are clamps, an example of such attachment means can be a hook 39 and a buckle 41, the buckle 41 being fastened into the hook 39 when the clamp is locked. To ensure improved handling of the whole microphysiological system (1, 3), the frame 5 and/or the lid (11, 13), preferably the lid (11, 13), exhibits a structure integrating said attachments means 39 (visible on FIGS. 3 and 5). Indeed, in the case where the lid (11, 13) shows such attachment means necessary for the workability of the locking means 15, it is easier to ensure the cleanability of such attachments means as they form a separate entity from the locking means 15 themselves.

For example, the elastic means 19 comprise one or more springs. With preference, one or more springs are one or more coil springs. Advantageously, the microphysiological system (1, 3) can comprise a setting means configured for adjusting the level of the high position of the support plate. Such setting means can be a mechanism to tighten or untighten the elastic means with a screw that is accessible via the frame. This offers the possibility to the user of choosing the intensity of the contact force (i.e., the pressure) that is going to be applied upon closure, notably in the case where more than two channels are to be stacked together within the microphysiological system (1, 3).

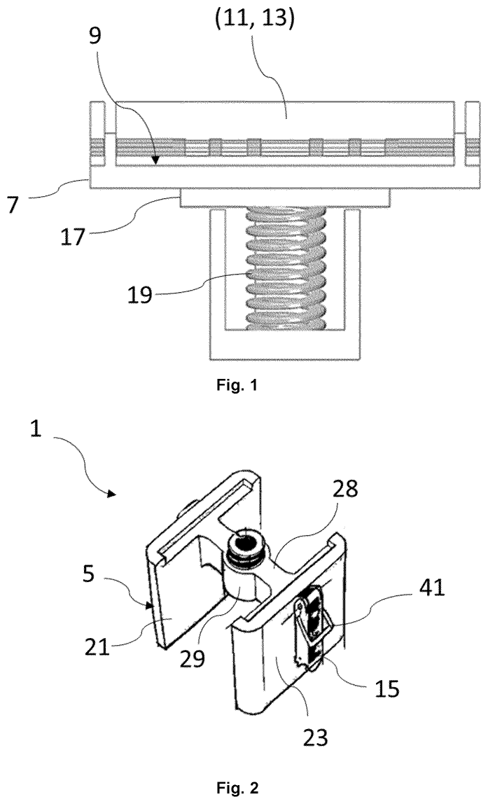

It is preferably that the elastic means 19 are arranged centrally with respect to the support plate 17 since symmetric arrangement favours a uniform distribution of the contact force upon closure. Thus, frame 5 can comprise guiding means 29 cooperating with the elastic means 19 and/or the support plate 17 to ensure a vertical movement of the support plate 17 with respect to frame 5. FIGS. 2 and 4 show that frame 5 has a structure presenting at least two vertical panels (21, 23) facing one another and connected by a connecting structure 28 wherein the connecting structure 28 presents one or more passages forming the guiding means 29.

FIG. 2 shows a particular microphysiological system 1 having two vertical panels (21, 23) connected by a connecting structure, leading subsequently to an open design of the microphysiological system 1. Such openings in frame 5 facilitate the installation of the tubings, since entrance means and exit means providing fluidic access to each channel are presented on each channel and subsequently reachable by the user from the exterior of the microphysiological system 1.

FIG. 3 shows the lid 11 that is associated with the microphysiological system 1 depicting an open design. The lid 11 for closing the microphysiological system 1 has a similar structure with at least two lateral elements (31, 33)—such as two bars—intending to cover at least a part of the top of the at least two vertical panels (21, 23) of the frame 5 and a central element 35. The central element 35 presents one or more passages 37 therein to receive the upper part of a spring as elastic means. The lid 11 for closing the microphysiological system 1 can also comprise one or more openings, for example from each side of the central element 35. These openings are suitable for performing optical analysis on the arrangement of said at least two or more channels (43, 45, 47, 49). With preference, at least a part of the one or more openings of the lid are covered with at least one optically transparent sheet made of an optically transparent plastic material.

The microphysiological system 3 depicted in FIG. 4 has, in addition to the two vertical panels (21, 23) and the connecting structure 28, auxiliary connecting structures (26, 30) binding the two side edges of each vertical panel (21, 23). In some implementations, only one auxiliary connecting structure can be present. The resulting configuration leads to a closed design or a semi-closed design in the case only one auxiliary connecting structure is present. The closed design requires that the tubing with the entrance means and exit means is done before closing the microphysiological system 3.

FIG. 5 shows the lid 13 that is associated with the microphysiological system 3 depicting a closed design. The lid 13 for closing the microphysiological system 3 has a similar structure as the frame 5, with at least two lateral elements (31, 33)—such as two bars-intended to cover at least a part of the top of the at least two vertical panels (21, 23) of the frame 5 and additional lateral elements (36, 40)—such as additional bars-intended to cover the auxiliary connecting structures (26, 30) binding the two side edges of each vertical panel (21, 23). It is therefore apparent that the lid for closing the microphysiological system 3 comprises an opening. Such an opening is useful for performing optical analysis of the arrangement of said at least two or more channels (43, 45, 47, 49). With preference, all or part of the opening of the lid is covered with at least one optically transparent sheet made of an optically transparent plastic material.

The fact that the lid (11, 13) that has a similar structure of the frame 5 ensures that the design of the lid (11, 13) is mostly cut-out, facilitating subsequently the accessibility to the microphysiological system (1, 3). The cut-out design of the lid (11, 13) leaves a wide area to the top surface of the underlying microphysiological system (1, 3). This wide area is accessible with any kind of suitable biological instruments, such as tubes and/or electrodes among others, necessary to the adequate handling of the microphysiological system (1, 3) of the present disclosure. This is of great interest especially because the configuration of the microphysiological system (1, 3) allows to exert a mechanical pressure on the device thanks to the locking means 15 fastening the frame 5 and the lid (11, 13) in order to reduce or prevent leakage.

FIG. 6 details the lid 13 associated with the microphysiological system 3 depicting a closed design. It can be seen that the lid 13 has a design with a least two edges facing each other, wherein several ports are provided. There are preferably one port 65 along one of said two edges of the lid 13, so that said port 65 can received a pin that is provided along the edge of the base 7. Advantageously, as the pins provided along the edge facing each other of the base 7 are shifted between each other, the ports 65 are also shifted between each other.

FIG. 6 also shows several additional ports 63 that are placed in the corner of the lid 13.

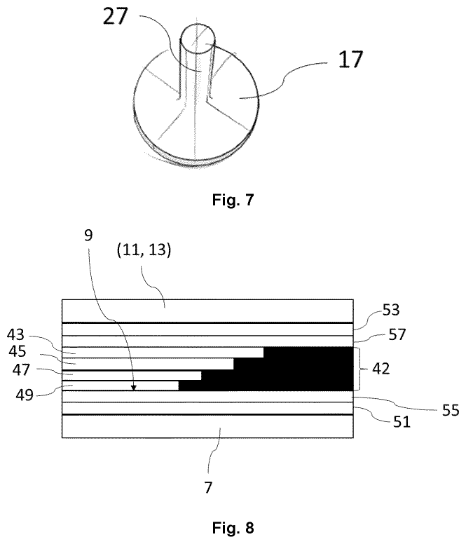

The support plate 17 can comprise one or more positioning means 27 cooperating with the elastic means 19 and/or the frame 5 to ensure a vertical movement of the support plate 17 with respect to the frame 5. With preference, as shown in FIG. 7, the positioning means 27 of the support plate 17 comprise one or more feet, more preferably one foot. In a preferred embodiment, a coil spring is coiled around a foot to ensure the vertical movement of the support plate 17.

During sterilization, the microphysiological system (1, 3) must endure relatively harsh conditions. Indeed, in addition to being assembled and disassembled many times, the sterilation are often conducted under autoclave conditions, namely under high-pressure and high-temperature conditions. For example, the pressure can range between 0.1 MPa and 0.5 MPa, preferably between 0.2 MPa and 0.4 MPa and/or the temperature can be of at least 90° C., preferably at least 100° C., more preferably at least 110° C. For example, the temperature can range between 90° C. and 300° C., preferably between 100° C. and 290° C. or between 110° C. and 280° C. To ensure the robustness of the microphysiological system (1, 3), the structure of the frame 5 and/or the lid (11, 13) is in a metallic material, for example, a metallic material selected from steel, titanium, aluminium, copper or a mixture thereof, preferably in steel. For example, the structure of frame 5 and/or of the lid (11, 13) can be in stainless steel. However, to ensure that optical analysis can be carried out, the whole structure of the frame 5 and/or of the lid (11, 13) is covered by optically transparent plastic material, that can be either polycarbonate or cyclic olefin copolymer, preferably polycarbonate. In other words, the base 7 of the frame 5and/or the support plate is made in such optically transparent plastic material.

The arrangement of said at least two or more channels (43, 45, 47, 49) is preferably a layer of optically transparent plastic material, such as polycarbonate or cyclic olefin copolymer, more preferably polycarbonate.

In a preferred embodiment, as shown in FIG. 8, there are at least four channels (43, 45, 47, 49) placed one above another. They can be arranged on top of each other in a staggered manner for allowing a fluidic connection via the entrance means and/or the exit means presented by the lid and/or the base with one end of the tubings that bring the necessary elements to maintain the cell culture alive. Alternatively, the edges of each channel are aligned as well as the surface of each channel and the channel differs between each other in that the location of the entrance means and/or the exit means for allowing the fluidic connection is different. In that case, these are the entrance means and/or the exit means on each channel that can be arranged in a staggered manner. The necessary elements, to maintain the cell culture alive, are stored in a storage place connected to the other end of the tubings. To this respect, each entrance means and exit means is a fluid connecter fitted with a taper with a length being equal to or inferior to the thickness of the lid and/or of the base of the frame. The fact that the length of the taper is not superior to the thickness of the lid and/or of the base of the frame allows to keep the lid and/or the base of the frame flat, facilitating, therefore, the handling of these components of the microphysiological system (1, 3) by the user. For example, the taper can be a Luer-type taper or a Barb-type taper, depending on how the microphysiological system (1, 3) is made. Thus, when the microphysiological system is milled, the taper is a Luer-type taper since the milling prevents the making of Barb-type tapers. Those one can be present only if the microphysiological system is made from a mold.

Each of the channels (43, 45, 47, 49) forms a chamber. The chamber is a free-space that can receive the cell culture and/or the elements that are necessary for the survival of the cells. For example, the chambers can be in the form of a spiral. In an alternative embodiment, the chambers have a zig-zag shape instead of a spiral, which faster removal of the air bubbles from the chamber. It is preferred that the chambers are devoid of an angular corner to avoid turbulent flow. In an alternative embodiment, the chambers are straight, facilitating thus the upstream and downstream automation with American National Standards Institute-Society for Biomedical Sciences (ANSI-SBS) standard plates which are the 96-well microplate standard.

When the cell culture support 42 comprises two channels, a first channel comprises a cell culture layer mimicking the vascular circuit and a second channel, on top of the first channel, comprises epithelium cells, which is perfused by a liquid, e.g., cell culture medium, using external pumps necessary for having a microfluidic activity. It is however preferred that the cell culture support 42 comprises four channels, namely a first channel comprising a cell culture layer mimicking the vascular circuit, a second channel comprising the epithelium layer, a third channel comprising the microbiome layer and a fourth channel with a chamber used for conducting the inert gas (i.e., nitrogen).

To provide access during operation to the chamber, the lid (11, 13) for closing the microphysiological system (1, 3) can have one threaded opening per channel (43, 45, 47, 49). During operation, the threaded opening is closed with a screw and can be opened. Once open, it is possible to collect a sample with a pipette. For example, the threaded opening has a diameter ranging between 5 mm and 15 mm, or between 6 mm and 10 mm. This can be one way by which the samples can be recovered for subsequent analysis.

To provide online monitoring of various parameters during operation, the lid (11, 13) for closing the microphysiological system (1, 3) can comprise one or more optodes selected for example from an oxygen optode, a carbon dioxide optode or a pH optode. With preference, all these three optodes are set up on the lid (11, 13). The one or more optodes must be smaller than the width of the channel to be able to accurately measures inside the liquid. In addition, to avoid leakage at the level of the optode, the width of the optode must be smaller than the maximum width of the chamber formed by the channel (43, 45, 47, 49).

Upon installation of the arrangement of the at least two channels within the microphysiological system (1, 3) and, in particular, during its closure, leakage can also occur when the channels are pressed on each other. To avoid this effect, the surface 9 exhibited by the base for receiving a cell culture support 42 can present recesses and the lid (11, 13) for closing the microphysiological system (1, 3) can have a stepped surface facing the surface 9. In other words, the lid (11, 13) presents one or more steps and the surface 9 presents one or more recesses. As the material from which the base and the lid are made (i.e., preferably the optically transparent plastic material) bends under the pressure of the locking means 15, the step-and-recess feature takes advantage of this property and avoids that the pressure applied upon closure warps the lid as well as the top of the base, subsequently preventing leakage upon closure. The sealing effect can also be reinforced by the use of an appropriate elastomer as a gasket, for example, silicone rubber (e.g., PolySil™ SA1020 or 12″×12″× 1/32″ Orange-red Silicone sheet—MSC #85987857). For example, the depth of a recess and/or the height of a step is greater than or equal to 0.05 mm and less than or equal to 0.3 mm. For example, the ratio of the depth of the recess to the thickness of the base 7 is ranging between 0.01 and 0.1. For example, the ratio of the height of the step to the thickness of the lid (11, 13) is ranging between 0.01 and 0.1.

Advantageously, the base 7 exhibiting the surface 9 for receiving a cell culture support 42 shows a poka-yoke design to facilitate the arrangement of the channel (43, 45, 47, 49) within the microphysiological system (1, 3). For example, each channel (43, 45, 47, 49) comprises at least two holes and the base 7 shows at least two pins oriented vertically. In that manner, the holes of the channel (43, 45, 47, 49) can be inserted into the pins of the base, and this improves the assembly of the microphysiological system (1, 3). This poka-yoke design is also particularly exemplified with the lid 13 depicted on FIG. 6, since in this configuration, there is a shift between the two ports 65 that are along two different edges of the lid 13, said two different edges facing each other. The resulting asymmetry ensures that the assembly of the microphysiological system (1, 3) is only possible with all the layers in their correct orientation. Indeed, the asymmetry ensures that inverted or rotated layers simply will not fit with the pins provided by the base 7. This at least reduces or even prevents any leakage problem, because as the layers could look quite similar to the users'eyes, their incorrect assembly would likely result in leakage. The poka-yoke design, provided by the shifted/asymmetrical pins and their corresponding ports 65 prevent this. This further allows to ensure the robust sealing of the microphysiological system (1, 3) since the correct orientation of all the elements, including the base 7, the lid (11, 13) and/or also the channels (43, 45, 47, 49), between each other must be respected thanks to this particular design. Handling of the microphysiological system (1, 3) is also considerably enhanced with this particular design.

It is possible to adapt the microphysiological system (1, 3) with an incubator function, by placing for example a first electrical heating mat 51 under the surface 9 for receiving a cell culture support 42 and a second electrical heating mat 53 under the lid (11, 13) for closing the microphysiological system. As shown in FIG. 8, it is advantageous that a first conduct 55 intended to be filled with a liquid, for example water, is arranged between the first electrical heating mat 51 and the surface 9 and that a second conduct 57 intended to be filled with a liquid, for example water, is arranged under the second electrical heating mat 53 to allow temperature sensing. The first and second conduct (55, 57) intended to be filled with a liquid are respectively adjacent to the first and second electrical heating mat (51, 53). For example, the second conduct 57 intended to be filled with a liquid is under the second electrical heating mat 53 and facing the cell culture support 42 of the base 7.

1. A microphysiological system (1, 3) comprising a frame (5) with a base (7) exhibiting a surface (9) for receiving a cell culture support (42), a lid (11, 13) for closing the microphysiological system (1, 3), and locking means (15) fastening the frame (5) and the lid (11, 13) when said microphysiological system (1, 3) is closed, the microphysiological system (1, 3) is characterized in that the base (7) is disposed within the frame (5) on a support plate (17) displaceable between a high position when the microphysiological system (1, 3) is open and a low position when the microphysiological system (1, 3) is closed, in that the support plate (17) is movable vertically between its high position and low position with elastic means (19) configured to bring the support plate (17) back to the high position, and in that the base (7) includes at least two pins oriented vertically and the lid (11, 13) comprises at least two ports (65) in which said at least two pins are arranged when said microphysiological system (1, 3) is closed.

2. The microphysiological system (1, 3) according to claim 1, characterized in that the base (7) includes at least two edges facing each other, and wherein the base presents one of said at least two pins oriented vertically along each of said two edges facing each other.

3. The microphysiological system (1, 3) according to claim 2, characterized in that said at least two pins oriented vertically are shifted between each other.

4. The microphysiological system (1, 3) according to claim 1, characterized in that the base (7) and the lid (11, 13) include each at least one access aperture.

5. The microphysiological system (1, 3) according to claim 1, characterized in that the elastic means (19) are or comprise one or more springs.

6. The microphysiological system (1, 3) according to claim 1, characterized in that the elastic means (19) are arranged centrally with respect to the support plate (17).

7. The microphysiological system (1, 3) according to claim 1, characterized in that the support plate (17) comprises one or more positioning means (27) cooperating with the elastic means (19) and/or with the frame (5) to ensure a vertical movement of the support plate (17) with respect to the frame (5).

8. (canceled)

9. The microphysiological system (1, 3) according to claim 1, characterized in that the frame (5) comprises one or more passages forming guiding means (29) cooperating with the elastic means (19) and/or the support plate (17) to ensure a vertical movement of the support plate (17) with respect to the frame.

10. The microphysiological system (1, 3) according to claim 1, characterized in that the frame (5) has a structure presenting at least two vertical panels (21, 23) facing one another and connected by a connecting structure (28) wherein the connecting structure (28) presents one or more passages forming guiding means (29).

11. (canceled)

12. (canceled)

13. The microphysiological system (1, 3) according to claim 1, characterized in that the locking means (15), fastening the frame (5) and the lid (11, 13), comprises attachment means (41) cooperating with hooks (39), wherein the hooks (39) are formed by an outgrowth of the lid (11, 13) or of the frame (5).

14. The microphysiological system (1, 3) according to claim 1, wherein the elastic means (19) is configured for adjusting the level of the high position of the support plate (17).

15. The microphysiological system (1, 3) according to claim 1, characterized in that the lid (11, 13) for closing the microphysiological system (1, 3) comprises one or more openings.

16. (canceled)

17. (canceled)

18. The microphysiological system (1, 3) according to claim 1, characterized in that the support plate is made of an optically transparent plastic material.

19. The microphysiological system (1, 3) according to claim 1, characterized in that the surface (9) exhibited by the base for receiving the cell culture support (42) is a recessed surface and in that the lid (11, 13) for closing the microphysiological system (1, 3) has a stepped surface facing the surface of the base (7).

20. The microphysiological system (1, 3) according to claim 1, characterized in that the microphysiological system (1, 3) comprises a first electrical heating mat (51) under the surface (9) for receiving the cell culture support (42) and a second electrical heating mat (53) under the lid (11, 13) for closing the microphysiological system (1, 3).

21. The microphysiological system (1, 3) according to claim 20, characterized in that the microphysiological system (1, 3) further comprises a first conduct (55) configured to be filled with liquid and arranged between the first electrical heating mat (51) and the surface (9) for receiving the cell culture support

(42) and a second conduct (57) configured to be filled with liquid and arranged under the second electrical heating mat (53).

22. (canceled)

23. The microphysiological system (1, 3) according to claim 1, characterized in that the microphysiological system (1, 3) further comprises the cell culture support (42) arranged on the surface (9), wherein the cell culture support (42) is an arrangement of at least two or more channels (43, 45, 47, 49), each channel forming a chamber and each channel being stacked one on the other.

24. The microphysiological system (1, 3) according to claim 23, characterized in that each of the channels (43, 45, 47, 49) comprises at least two holes in which said at least two pins are arranged.

25. The microphysiological system (1, 3) according to claim 23, characterized in that the channels (43, 45, 47, 49) are placed on top of one another in a staggered manner.

26. The microphysiological system (1, 3) according to claim 23, characterized in that each channel is a straight channel.

27-31. (canceled)
